(19) United States
(12) Patent Application Publication
(10) Pub. No.: US 2021/0196448 A1
(43) Pub. Date: Jul. 1, 2021

(54) Tendon Interference Anchor

(71) Applicant: Acuitive Technologies, Inc., Allendale, NJ (US)

(72) Inventors: Rui J. Ferreira, Livingston, NJ (US); Christopher C. Larson, Lincoln Park, NJ (US); Michael P. McCarthy, Ho-ho-kus, NJ (US); Douglas William Gabel, Pompton Plains, NJ (US)

(73) Assignee: Acuitive Technologies, Inc., Allendale, NJ (US)

(21) Appl. No.: 17/271,421

(22) PCT Filed: Aug. 27, 2019
(86) PCT No.: PCT/US2019/048313

§ 371 Date: Feb. 25, 2021

Related U.S. Application Data
(60) Provisional application No. 62/725,831, filed on Aug. 31, 2018.

(51) Int. Cl. A61F 2/08

(52) U.S. Cl. CPC A61F 2/0811; A61F 2002/0841; A61F 2002/0858; A61F 2230/0067; A61F 2210/0004; A61F 2220/0016; A61F 2002/0888

(57) ABSTRACT

The present disclosure provides advantageous interference anchor. More particularly, an advantageous tendon interference anchor configured to be inserted through impaction. The tendon interference anchor may include an anchor body having a proximal end, distal end, longitudinal axis, and an outer surface and at least one protrusion extending outwardly from the outer surface of the anchor body in a direction non-parallel to the longitudinal axis of the anchor body.

### CROSS-REFERENCE TO RELATED APPLICATION

The present application claims priority benefit to a U.S. provisional patent application entitled “Tendon Interference Anchor,” which was filed on Aug. 31, 2018, and assigned Ser. No. 62/725,831. The content of the foregoing provisional application is incorporated herein by reference.

### FIELD OF THE DISCLOSURE

The present disclosure generally relates to interference anchors and, more generally, a tendon interference anchor inserted through impaction.

### BACKGROUND OF THE DISCLOSURE

Traditional tendon interference anchors are torsionally installed into a desired fixation location (e.g., soft or hard tissue). Traditional installation methods impart increased torsion and tensile stresses on the material of the desired fixation location. Additionally, the constant sliding of the thread major of the anchor along a tendon and bone creates friction and further increases the probability of lacerating a tendon.

Based on the foregoing, a need exists for an effective anchor that will reduce the probability of anatomical damage. Thus, an interest exists for improved tendon interference anchors, and related methods of use. These and other inefficiencies and opportunities for improvement are addressed and/or overcome by the assemblies, systems and methods of the present disclosure.

### SUMMARY OF THE DISCLOSURE

The present disclosure provides an advantageous system for improved fixation with soft tissue (e.g., tendon) and/or hard tissue (e.g., bone). In particular, the present disclosure is directed to systems/methods that incorporate a tendon interference anchor with advantageous protrusions (e.g., splines, I-beams, barbs, knurls, threads, ribs, ridges, tines, teeth, wedges, fins, grooves, and any combination thereof) to affix soft tissue, medical implants (e.g., bone plates), and/or hard tissue to hard tissue. Even more particularly, exemplary systems/methods are disclosed that include an anchor system having an anchor body and protrusions for securing the anchor to the desired fixation location (e.g., desired bone location). The disclosed anchor system is configured to be advanced and fixed into a desired fixation location through impaction.

In exemplary embodiments, the present disclosure provides an advantageous anchor system for improved fixation between a variety of anatomical surfaces and/or structures (e.g., soft tissue, hard tissue, and/or medical implants). The disclosed anchor system includes an anchor body having a proximal end, a distal end, a longitudinal axis, an outer surface and at least one protrusion (e.g., one or more splines, I-beams, barbs, knurls, threads, ribs, ridges, tines, teeth, wedges, fins, grooves, and any combination thereof) for securing the anchor system relative to a desired fixation location. The outer surface of the anchor body may include the at least one protrusion extending outwardly therefrom in a direction non-parallel to the longitudinal axis of the anchor body. Further, the at least one protrusion may extend radially from the longitudinal axis.

The disclosed anchor system may be advantageously inserted into the desired fixation location through impaction. Depending on the protrusion style, a slight rotation of the anchor system may occur during the impaction. In some embodiments, the rotation assists the impaction to further secure the anchor relative to the surrounding anatomical surfaces and/or structures (e.g., soft and hard tissue). In those instances, it is understood that the impaction forces are the primary and substantial driver in inserting/advancing the anchor into the desired fixation location. In other embodiments, the impaction forces and the resultant rotational forces work in unison to insert/advance the anchor into the desired fixation location.

In an exemplary embodiment, an advantageous anchor system includes an anchor body having a proximal end, a distal end, a longitudinal axis, an outer surface and at least one helical spline associated, in whole or in part, with the outer surface of the disclosed anchor body. In exemplary embodiments, the disclosed anchor may include 5 or 6 external helical splines having a twist angle ranging from about 180 degrees to about 360 degrees. However, more or less external helical splines may be utilized without departing from the scope or spirit of this disclosure. The disclosed anchor system may further include an enlarged head to limit the depth that the anchor system may be impacted and/or travel relative to the desired fixation location. The disclosed anchor system may further include at least one (or both) of a cannulation hole and a cross hole. The disclosed hole(s) may enable an in situ settable polymer to enter and/or pass through the anchor body.

In another exemplary embodiment, an advantageous anchor system includes an anchor body having a proximal end, a distal end, a longitudinal axis, an outer surface and at least one helical spline associated, in whole or in part, with the outer surface of the disclosed anchor body. The disclosed anchor body may further include a plurality of protrusions extending, in whole or in part, from one or more of the disclosed splines. The disclosed anchor system may further include at least one (or both) of a cannulation hole and a cross hole. A four strand graft, previously secured to a femur, may be pulled through the cannulation hole. The strands may be separated and positioned within each quadrant of the disclosed anchor.

In yet another embodiment, an advantageous anchor system includes an anchor body having a proximal end, a distal end, a longitudinal axis, an outer surface and at least one spline associated, in whole or in part, with the outer surface of the disclosed anchor body. The disclosed spline(s) may function, inter alia, to vary the amount of potential engagement with the disclosed fixation location, such that at one position along the anchor body the disclosed spline(s) may engage with the opposing hard/soft tissue more than at another position along the anchor body. The disclosed anchor may further include a plurality of protrusions at least partially positioned along one or more splines. The disclosed anchor system may further include at least one (or both) of a cannulation hole and a cross hole. In some instances, a four strand graft, already secured to a femur, may be pulled through the cannulation hole. The strands are separated and positioned within each quadrant of the depicted anchor body.

Any combination or permutation of features, functions and/or embodiments as disclosed herein is envisioned. Additional advantageous features, functions and applications of the disclosed systems, methods and assemblies of the present disclosure will be apparent from the description which follows, particularly when read in conjunction with the appended figures. All references listed in this disclosure are hereby incorporated by reference in their entireties.

### BRIEF DESCRIPTION OF DRAWINGS

Features and aspects of embodiments are described below with reference to the accompanying drawings, in which elements are not necessarily depicted to scale.

Exemplary embodiments of the present disclosure are further described with reference to the appended figures. It is to be noted that the various features, steps and combinations of features/steps described below and illustrated in the figures can be arranged and organized differently to result in embodiments which are still within the scope of the present disclosure.

To assist those of ordinary skill in the art in making and using the disclosed assemblies, systems and methods, reference is made to the appended figures, wherein:

FIGS. 1A-1B schematically depict an exemplary tendon interference anchor according to the present disclosure;

FIGS. 2A-2B schematically depict an exemplary tendon interference anchor according to the present disclosure;

FIGS. 3A-3D schematically depict exemplary tendon interference anchors according to the present disclosure;

FIG. 4 schematically depicts an exemplary tendon interference anchor according to the present disclosure;

FIG. 5 schematically depicts an assembled view of an exemplary tendon interference anchor and a desired fixation location, according to the present disclosure;

FIGS. 6A-6B schematically depict an exemplary tendon interference anchor according to the present disclosure;

FIG. 7 schematically depicts an exemplary tendon interference anchor according to the present disclosure;

FIG. 8 schematically depicts an exemplary tendon interference anchor according to the present disclosure; and

FIG. 9 schematically depicts an exemplary tendon interference anchor according to the present disclosure.

### DETAILED DESCRIPTION OF DISCLOSURE

The exemplary embodiments disclosed herein are illustrative of advantageous systems for improved fixation with soft tissue (e.g., tendon) and/or hard tissue (e.g., bone). It should be understood, however, that the disclosed embodiments are merely illustrative of the present disclosure, which may be embodied in various forms. Therefore, details disclosed herein with reference to exemplary assemblies/fabrication methods and associated processes/techniques of assembly and use are not to be interpreted as limiting, but merely as the basis for teaching one skilled in the art how to make and use the advantageous assemblies/systems of the present disclosure.

The present disclosure provides an advantageous system/method of an exemplary tendon interference anchor with improved protrusions to affix soft tissue, medical implants (e.g., bone plates), and/or hard tissue to hard tissue. Even more particularly, exemplary systems/methods are disclosed which include an anchor system having an anchor body and protrusions for securing the anchor to the desired bone location. The disclosed anchor system is configured to be substantially impacted into the desired bone location. Particularly, where the primary form of insertion is impaction. As used herein, tendon interference anchor may be referred to as “interference anchor,” “anchor system,” or “anchor”.

The disclosed tendon interference anchor may include an anchor body and at least one protrusion (e.g., splines, I-beams, barbs, knurls, threads, ribs, ridges, tines, teeth, wedges, fins, grooves, and any combination thereof). At least one protrusion is at least partially associated with the outer surface of anchor body. Anchor body and at least one protrusion may be fabricated as a single component or may be two separate components that are assembled together to give the appearance/functionality of a single component. The present disclosure is not intended to be limiting and the use of “anchor body” and “protrusion” are merely explanatory, therefore, both fabrication variations are anticipated, unless expressly stated otherwise.

The disclosed anchor has a proximal end, a distal end, and a longitudinal axis. Distal end may include at least one element to assist with the insertion of anchor into the desired fixation location. For example, anchor may include a substantially conical distal tip. However, distal tip may be fabricated into a variety of shapes, including, cylindrical, spherical, and/or with a loop, which is adapted for retention of at least one suture or cable. The disclosed anchor may have an anchor body with a cross-section that is substantially circular. However, the disclosed anchor body is not limited to the disclosed cross-section and a variety of shapes may be utilized (e.g., an oval, an ellipse, a quadrilateral, a triangle, or a combination thereof).

The disclosed anchor system may further include at least one of a cannulation hole and a cross hole. In some instances, a four strand graft, already secured to a femur, may be pulled through the cannulation hole. The strands may be separated and positioned within each quadrant of the depicted anchor. The disclosed anchor system may further include an element located in relation to the proximal end that is at least partially larger than the cross-section of the anchor system (e.g., the head of a nail). However, in other instances, the element may not exceed the cross-section of the anchor system in all directions. The disclosed anchor system may be fabricated into any desired length, as will be appreciated by one skilled in the art based on the anticipated use.

The disclosed anchor may be introduced into a desired fixation location (e.g., soft tissue and/or hard tissue). As used herein, the desired fixation location may be a desired position, located in close proximity to an anatomical surface, or may be at least a partial hole wherein the disclosed anchor may be introduced. In one embodiment, the desired fixation location includes a hole having a diameter that is substantially similar to the diameter of the disclosed anchor body. As previously stated, the disclosed anchor may be introduced into the desired fixation location through impaction. Depending on the desired protrusion style and orientation, the disclosed anchor may rotate during impaction. However, in terms of the primary insertion of the anchor, rotation is ancillary to impaction. Rotation, as a secondary form of insertion, helps to secure the disclosed anchor to the desired fixation location, without the disclosed pitfalls of torsional insertion.

In operation, an impactive force may be applied directly/indirectly to the proximal end of the disclosed anchor. In an exemplary embodiment, where the disclosed anchor includes helical protrusions, the disclosed anchor may slightly rotate during impaction into the desired fixation location. However, in another exemplary embodiment, where the disclosed anchor includes non-helical protrusions, the disclosed anchor may be impactively inserted into the desired fixation location without ancillary rotation. The desirable method depends on the application.

The disclosed anchor may be at least partially fabricated from a biodegradable citrate-based composite. The disclosed anchor may be bioabsorbable. The disclosed anchor system may be at least partially formed from the polycondensation product of citric acid and/or citrate with at least one C₂ to C₂₀ alkane diol. More particularly, the disclosed anchor system may be at least partially formed from the polycondensation product of citric acid and/or citrate with at least one C₄ to C₁₂ alkane diol. In certain embodiments, the citrate-based (co)polyester may be poly(1,8-octanediol citrate). In certain embodiments, the disclosed anchor may at least in part be formed from a composite comprising a citrate-based polymer and a bioceramic. In certain embodiments, the bioceramic is selected from the group including hydroxyapatite and beta-tricalcium phosphate. The citrate-based polymer(s) and the bioceramic(s) may be present in the composite in any suitable weight ratio relative to each other.

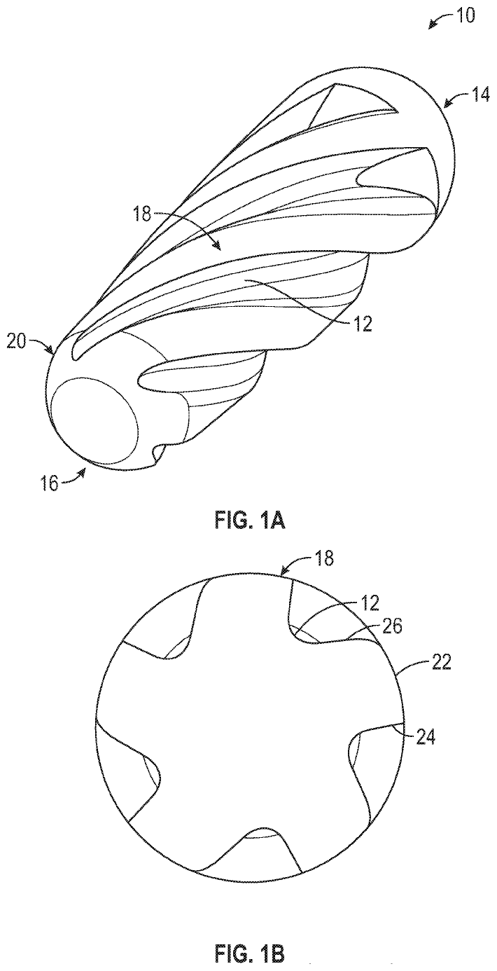

Examples of the tendon interference anchors and anchor systems/methods according to the present disclosure are illustrated in FIGS. 1-9.

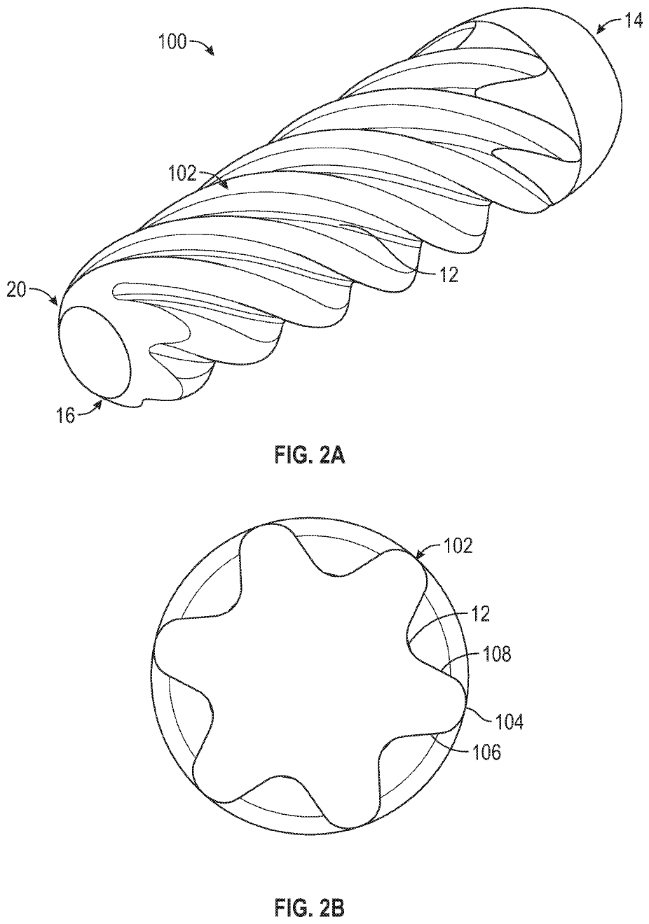

With reference to FIGS. 1A-2B, exemplary tendon interference anchor 10, 100 includes anchor body 12 and at least one protrusion 18, 102 (e.g., splines, I-beams, barbs, knurls, threads, ribs, ridges, tines, teeth, wedges, fins, grooves, and any combination thereof). Anchor body 12 may be substantially solid, partially solid, or substantially hollow. At least one protrusion 18, 102 is at least partially associated with the outer surface of anchor body 12. Anchor 10, 100 has a proximal end 14 and a distal end 16, and a longitudinal axis. Distal end 16 may include at least one element to assist with the insertion of anchor 10 into the desired fixation location. For example, anchor 10, 100 may include a substantially conical distal tip 20. Anchor body 12 may have a cross-section that is substantially circular, as illustrated in FIGS. 1B and 2B.

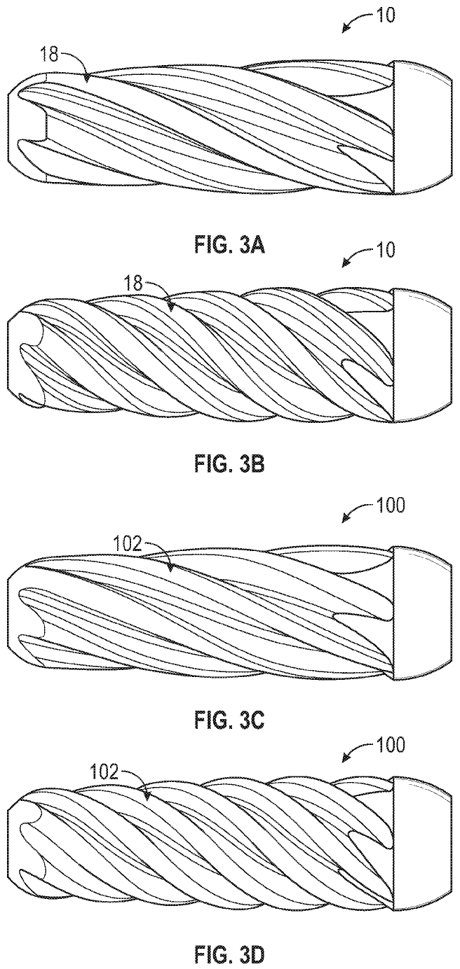

In an exemplary embodiment, at least one protrusion 18, 102 may be a spline. More particularly, the disclosed spline 18, 102 may be helical having a twist angle ranging from about 180 degrees to about 360 degrees. The disclosed helical splines 18, 102 may begin in close proximity to proximal end 14 and end in close proximity to distal end 16. In some examples, the disclosed helical splines 18, 102 may twist from about proximal end 14 to about distal end 16. Spline 18, 102 may extend outwardly from the outer surface of anchor body 12 in a direction non-parallel to the longitudinal axis of anchor body 12. As illustrated in FIG. 1A, helical spline 18 has a twist angle of about 180 degrees. In comparison, as illustrated in FIG. 2A, helical spline 102 has a twist angle of about 360 degrees. The disclosed anchor 10, 100 may include five helical splines 18, as depicted in FIG. 1B, or may include six helical splines 102, as depicted in FIG. 2B. Although depicted as having five helical splines 18 and a twist angle of about 180 degrees and six helical splines 102 and a twist angle of about 360 degrees, several variations may be appreciated, as depicted in FIG. 3, discussed below. The design of at least one protrusion 18, 102 may be optimized depending on the desired fixation location (e.g., soft tissue or hard tissue). Particularly, at least one protrusion 18, 102 may be configured and adapted to provide grip to the desired fixation location and to resist (or substantially resist) motion in at least one direction. For example, resisting (or substantially resisting) rotational movement, translational movement, and/or longitudinal movement.

The design of spline 18, 102 may be substantially symmetrical (e.g., similar to the cross-section of a V-shaped thread, a rounded V-shaped thread, a square thread, an acme thread, a knuckle thread, among others), such that bearing surface (i.e., engaging surface) 106 and opposing thread surface 108 have a substantially similar angle (i.e., spline/thread angle) in relation to the longitudinal axis of anchor body 12. (See, e.g., FIG. 2B). It should be appreciated, however, that the spline angle may be altered without departing from the spirit/scope of this disclosure. Therefore, the spline angle is not limited to those angles used in common applications (e.g., interfacing with metal, wood, plastic, or a bolt/nut configuration).

Alternatively, the design of spline 18, 102 may be biased (e.g., biased in a downward direction) so as to limit movement of the engaged anatomical surface(s) (e.g., tendon). (See, e.g., FIG. 1B). The bearing surface (i.e., engaging surface) 24 may be substantially perpendicular (or slightly biased/slanted) with reference to the longitudinal axis of anchor body 12. Opposing thread surface 26 may be a standard angle (e.g., 30, 45, 60, e.g., as will be known to a person skilled in the art). Designation of bearing surface 24, 106 and opposing thread surface 26, 108 is dependent on handedness of helical splines 18, 102.

In the present examples, helical splines 18, 102 are described having a right-handed configuration. However, a left-handed configuration is possible. In such instance, bearing surface 24, 106 and opposing thread surface 26, 108 would be switched. The depth of spline 18, 102, which is defined as the distance between outer surface 22, 104 of spline 18, 102 and the outer surface of anchor body 12, may at least partially affect the level of engagement between anchor 10, 100 and the desired fixation location. Much like the spline angle, the spline depth may be altered depending on the application and desired fixation location.

As previously discussed, five helical spline anchors and six helical spline anchors may each have a twist angle ranging from about 180 degrees to about 360 degrees. As previously illustrated, and reproduced as FIG. 3A for ease of comparison, anchor 10 has five helical splines 18 with a twist angle of about 180 degrees. FIG. 3B utilizes a similar five helical spline anchor 10, but with a twist angle of about 360 degrees. Additionally, as previously illustrated, and reproduced as FIG. 3D for ease of comparison, anchor 100 has six helical splines 102 with a twist angle of about 360 degrees. FIG. 3C utilizes a similar six helical spline anchor 100, but with a twist angle of about 180 degrees.

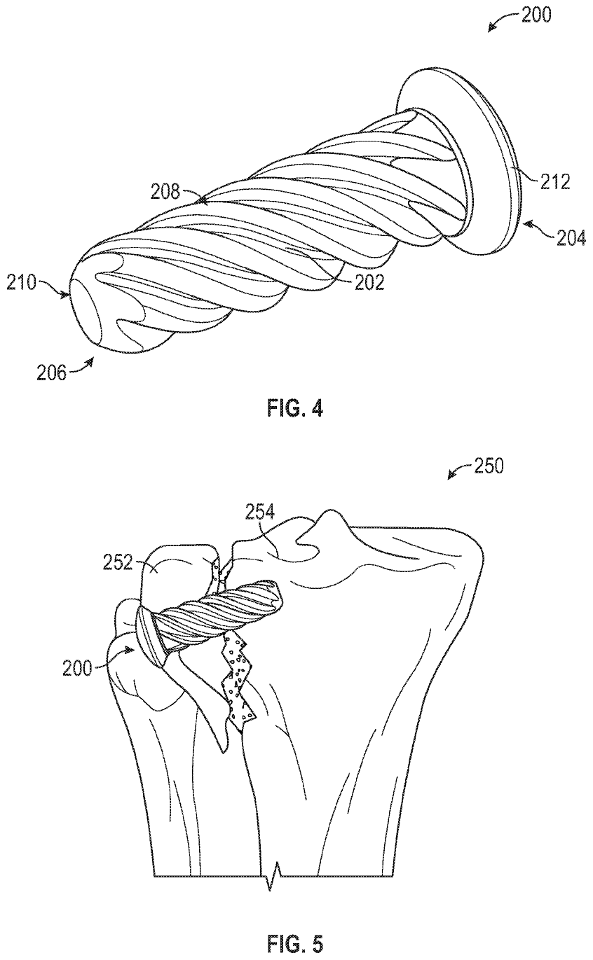

In another exemplary embodiment, as illustrated in FIG. 4, tendon interference anchor 200 includes anchor body 202 and at least one protrusion 208 (e.g., splines, I-beams, barbs, knurls, threads, ribs, ridges, tines, teeth, wedges, fins, grooves, and any combination thereof). Anchor 200 has a proximal end 204, a distal end 206, and a longitudinal axis. Anchor body 202 may be substantially solid, partially solid, or substantially hollow. At least one protrusion 208 is at least partially associated with the outer surface of anchor body 202. In some embodiments, at least one protrusion 208 may be a spline. In one example, anchor 200 includes six splines, as illustrated in FIG. 4. However, five splines may be utilized, as described in more detail above. More particularly, the disclosed spline(s) 208 may be helical having a twist angle ranging from about 180 degrees to about 360 degrees. In some examples, the disclosed helical splines 208 may twist from about proximal end 204 to about distal end 206.

Proximal end 204 may include mounting element (e.g., head) 212, configured and adapted to ensure at least a portion of anchor 200 remains in close proximity to the opening of the desired fixation location. More particularly, element 212 may ensure at least a portion of anchor 200 is external to, flush with, or slightly recessed to (e.g., countersink or counterbore) the opening of the desired fixation location. Element 212 may have a cross-section that is at least partially larger than the combined cross-section of at least one protrusion 208 and anchor body 202. Even more particularly, in an example where anchor 200 is joining two or more bone sections, discussed in more detail below with reference to FIG. 5, element 212 may ensure at least a portion of anchor 200 is external to, flush with, or slightly recessed to (e.g., countersink or counterbore) one of the bone sections.

In one example, as depicted in FIG. 4, element 212 may be similar to the head of a nail, screw, or the like. The cross-section of element 212 may be dimensioned such that at least a portion of element 212 may not exceed the cross-section of protrusion 208. For example, anchor body 202 may be T-shaped, wherein the vertical portion of the “T” is anchor body 202 and the cross portion, perpendicularly positioned atop anchor body 202, is element 212. Distal end 206 may include at least one element to assist with the insertion of anchor 200 into the desired fixation location. For example, anchor may include substantially conical distal tip 210. Anchor body 202 may have a cross-section that is substantially circular, as illustrated in FIGS. 1B and 2B. The iterations associated with FIGS. 1A-3D may further incorporate element 212 and, vice versa, anchor 200 may further incorporate features discussed above with reference to FIGS. 1A-3D. Therefore, it should be understood that the present disclosure is not rigidly defined by the figures and utilization of features from other figures is anticipated and encouraged.

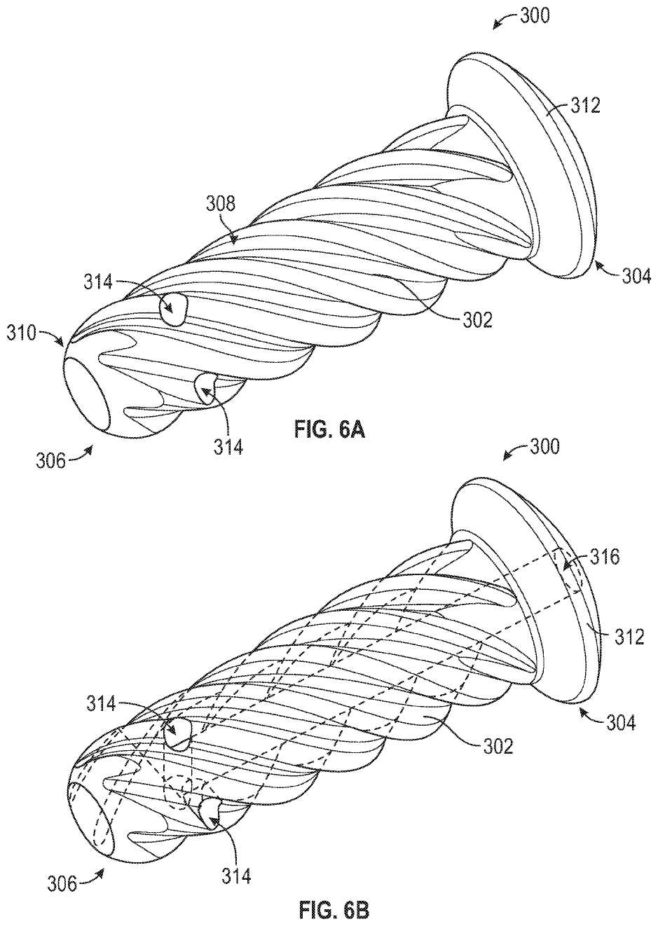

In another exemplary embodiment, as illustrated in FIGS. 6A-6B, tendon interference anchor 300 includes anchor body 302 and at least one protrusion 308 (e.g., splines, I-beams, barbs, knurls, threads, ribs, ridges, tines, teeth, wedges, fins, grooves, and any combination thereof). Anchor 300 has a proximal end 304, a distal end 306, and a longitudinal axis. Anchor body 302 may be substantially solid, partially solid, or substantially hollow. At least one protrusion 308 is at least partially associated with the outer surface of anchor body 302. In some embodiments, at least one protrusion 308 may be a spline. In one example, anchor 300 includes six splines, as illustrated in FIG. 6A. However, five splines may be utilized, as described in more detail above. More particularly, the disclosed spline(s) 308 may be helical having a twist angle ranging from about 180 degrees to about 360 degrees. In some examples, the disclosed helical splines 308 may twist from about proximal end 304 to about distal end 306.

Anchor 300 may include at least one hole (e.g., cannulation hole and cross hole). In an exemplary embodiment, anchor 300 may include at least one cross hole 314 and at least one cannulation hole 316. Cross hole 314 may extend at least partially through anchor body 302 at a desired non-parallel angle in relation to the longitudinal axis of anchor 300. For example, cross hole 314 may be substantially perpendicular to the longitudinal axis of anchor 300, as depicted in FIGS. 6A-6B. Anchor 300 may include two cross holes 314. As depicted, two cross holes 314 may be positioned substantially perpendicular to the longitudinal axis of anchor 300 and substantially perpendicular to each other. However, it should be understood that any orientation may be utilized. For example, at least two cross holes 314 may be positioned at different locations along the longitudinal axis of anchor 300. Anchor 300 may include cannulation hole 316 which extends at least partially through anchor body 300. For example, cannulation hole 316 may extend from proximal end through at least a portion of anchor body 302. In one example, cannulation hole 316 may extend along the longitudinal axis of anchor 300. Cannulation hole 316 may at least partially intersect with cross hole 314, as depicted in FIG. 6B.

In an exemplary embodiment, cannulation hole 316 extends from proximal end of anchor 300 at least partially through anchor body 302 along the longitudinal axis of anchor 300. At least two cross holes 314 may intersect cannulation hole 316. Cross holes 314 may be substantially perpendicular to cannulation hole 316 and may be substantially perpendicular to each other. Cross hole(s) 314 and cannulation hole 316 may facilitate interdigitation between an in situ polymer material (e.g., low viscosity) and the surrounding anatomy. In one example, anchor 300 may be inserted into the desired fixation location, as described above. Once inserted, an in situ polymer material may be introduced through cannulation hole 316. The in situ polymer material flows through cannulation hole 316 and out at least one cross hole 314, thereby interdigitating with the surrounding anatomy within desired fixation location. In another example, prior to insertion of anchor 300, the in situ polymer material may be introduced into the desired fixation location. During installation, anchor 300 compresses the in situ polymer material within the desired fixation location such that the in situ polymer material interdigitates the desired fixation location and anchor 300 (e.g., at least partially through cross hole 314 and/or cannulation hole 316).

In another example, anchor 300 may further include mounting element 312 (e.g., head), configured and adapted to ensure at least a portion of anchor 300 remains in close proximity to the opening of the desired fixation location. Element 312 may be associated with proximal end 304. More particularly, element 312 may ensure at least a portion of anchor 300 is external to, flush with, or slightly recessed to (e.g., countersink or counterbore) the opening of the desired fixation location. Element 312 may have a cross-section that is at least partially larger than the combined cross-section of at least one protrusion 308 and anchor body 302. Even more particularly, in an example where anchor 300 is joining two or more bone sections, element 312 may ensure at least a portion of anchor 300 is external to, flush with, or slightly recessed to (e.g., countersink or counterbore) one of the bone sections. (See, e.g., FIG. 5). Cannulation hole 316 may extend at least partially through element 312. Distal end 306 may include at least one element to assist with the insertion of anchor 300 into the desired fixation location. For example, anchor may include substantially conical distal tip 310. Anchor body 302 may have a cross-section that is substantially circular.

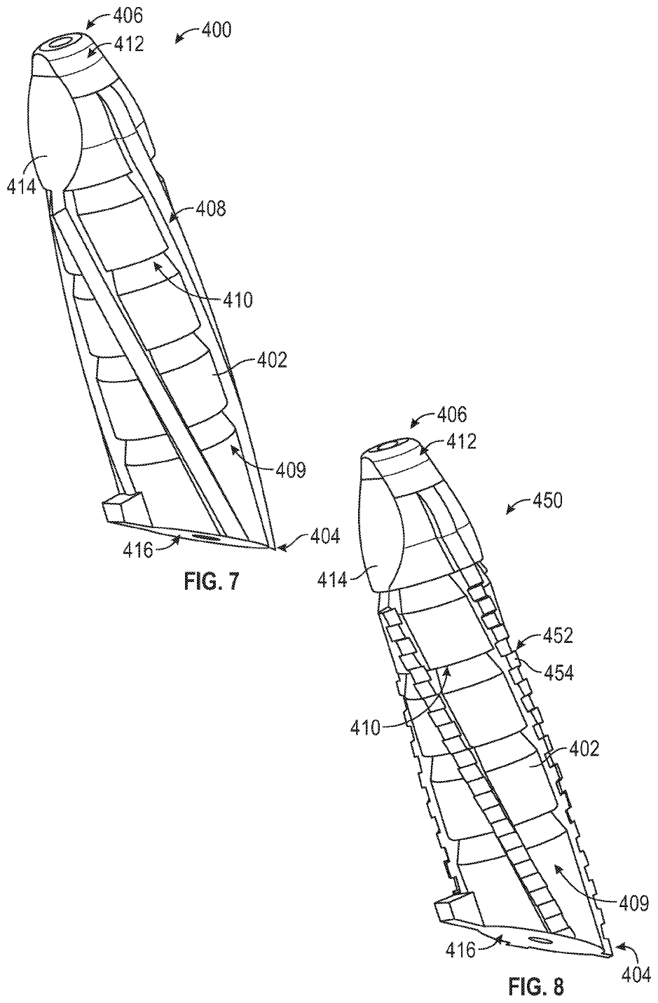

In another exemplary embodiment, as illustrated in FIGS. 7 and 8, tendon interference anchor 400 includes anchor body 402 and at least one protrusion 408 (e.g., splines, I-beams, barbs, knurls, threads, ribs, ridges, tines, teeth, wedges, fins, grooves, and any combination thereof). Anchor 400 has a proximal end 404, a distal end 406, and a longitudinal axis. Anchor body 402 may be substantially solid, partially solid, or substantially hollow. At least one protrusion 408, 452 is at least partially associated with the outer surface of anchor body 402. In some embodiments, at least one protrusion 408 may be a spline. Particularly, anchor 400 may include 4 protrusions 408, 452 (e.g., spline). More particularly, the disclosed spline(s) 408 may be helical having a twist angle ranging from about 1 degree to about 360 degrees. In an exemplary embodiment, the disclosed spline(s) 408, 452 may have a twist angle of about 12.9 degrees. Spline 408 may extend outwardly from the outer surface of anchor body 402 in a direction non-parallel to the longitudinal axis of anchor 400. In some examples, the disclosed helical splines 408 may twist from about proximal end 404 to about distal end 406. Between each spline 408 is groove 409. Groove 409 is defined by adjacent splines 408, 452 and anchor body 402.

Anchor 400 may further include at least one notch 410 that at least partially surrounds anchor body 402. Notch 410 may be designed such that removal of anchor 400 from the desired fixation location is more difficult than during insertion of anchor 400. Notch 410 may include a section that is angled with reference to the longitudinal axis of anchor 400. Particularly, notch 410 includes a section that is substantially perpendicular to the longitudinal axis of anchor 400. In an exemplary embodiment, anchor includes 4 notches 410 that at least partially surround anchor body 402. Notch 410 may facilitate interdigitation between an in situ polymer material (e.g., low viscosity) and the surrounding anatomy. In one example, prior to insertion of anchor 400 into the desired fixation location, the in situ polymer material may be introduced into the desired fixation location. During installation of anchor 400, anchor 400 compresses the in situ polymer material within the desired fixation location such that the in situ polymer material interdigitates the desired fixation location and anchor 400 (e.g., at least partially in relation to notch 410).

Anchor body 402 may further include hole 416 that at least partially extends from proximal end 404 towards distal end 406. In an exemplary embodiment, hole 416 extends from proximal end 404 through distal end 406 such that hole 416 is a through hole. Hole 416 may at least partially extend from proximal end 404 towards distal end 406 along the longitudinal axis of anchor 400. In one embodiment, hole 416 may facilitate interdigitation between an in situ polymer material and the surrounding anatomy, as discussed above. In another embodiment, hole 416 may at least partially engage with a graft (not shown). Particularly, in an instance where the graft (e.g., four strand graft) is already secured to the desired fixation location (e.g., femur), the graft may be pulled through hole 416. The strands of the graft may be separated and laid within a respective groove 409. Anchor 400 is inserted into the desired fixation location (e.g., through impaction) while pulling the graft to ensure proper tension.

Proximal end 404 may include a mounting element (e.g., head), configured and adapted to ensure at least a portion of anchor 400 remains in close proximity to the opening of the desired fixation location. More particularly, the mounting element may ensure at least a portion of anchor 400 is external to, flush with, or slightly recessed to (e.g., countersink or counterbore) the opening of the desired fixation location. The element may have a cross-section that is at least partially larger than the combined cross-section of at least one protrusion 408 and anchor body 402. Even more particularly, in an example where anchor 400 is joining two or more bone sections, the element may ensure at least a portion of anchor 400 is external to, flush with, or slightly recessed to (e.g., countersink or counterbore) one of the bone sections. Distal end 406 may include at least one element to assist with the insertion of anchor 400 into the desired fixation location. For example, anchor may include substantially conical distal tip 412. Conical distal tip 412 may further include surface 414, which may facilitate the separation of at least one strand from a graft (e.g., four strand graft). In a particular instance, surface 414 may be partially angled and slightly concaved. In one embodiment, surface 414 may separate two strands may from a four strand graft. The separated strands may be laid within a respective groove 409 of protrusion 408, 452, as mentioned above. Anchor body 402 may have a cross-section that is substantially circular.

In another exemplary embodiment, as specifically depicted in FIG. 8, the disclosed protrusions may further include at least one additional protrusion (e.g., barbs, knurls, threads, ribs, ridges, tines, teeth, wedges, fins, grooves, and any combination thereof) positioned thereon. Anchor 450 is a variation of anchor 400 and like features are numbered with the same reference numerals. Anchor 450 includes at least one protrusion 452 (e.g., splines, I-beams, barbs, knurls, threads, ribs, ridges, tines, teeth, wedges, fins, grooves, and any combination thereof), as discussed above. Protrusion 452 may be a helical spline. Positioned on an outer surface of protrusion 452 is at least one second protrusion 454 (e.g., barbs, knurls, threads, ribs, ridges, tines, teeth, wedges, fins, grooves, and any combination thereof). Protrusion 452 may extend outwardly from the outer surface of anchor body 402 in a direction non-parallel to the longitudinal axis of anchor body 402. In some examples, the disclosed helical splines 452 may twist from about proximal end 404 to about distal end 406. In one example, a plurality of second protrusions 454 may be positioned along spline 452. Various second protrusions 454, as outlined above, may be used depending on the desired fixation location (e.g., soft bone and/or hard bone).

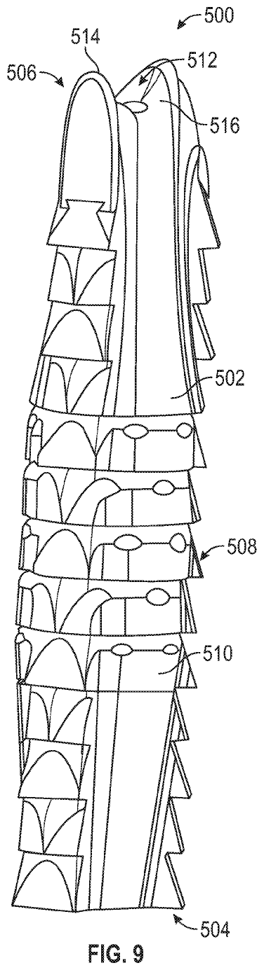

In yet another exemplary embodiment, as depicted in FIG. 9, tendon interference anchor 500 includes anchor body 502 and at least one protrusion 508 (e.g., splines, I-beams, barbs, knurls, threads, ribs, ridges, tines, teeth, wedges, fins, grooves, and any combination thereof). Anchor 500 has a proximal end 504, a distal end 506, and a longitudinal axis. Anchor body 502 may be substantially solid, partially solid, or substantially hollow. At least one protrusion 508 is at least partially associated with the outer surface of anchor body 502. In one example, a plurality of protrusions 508 are positioned on the outer surface of anchor body 502. Protrusions 508 may be configured such that the cross-sectional diameter of anchor 500 varies at different positions along the longitudinal axis of anchor 500. For example, the middle portion of anchor 500 may have a larger cross-sectional diameter than at least one end portion of anchor 500. Such dimensional variation provides differing levels of engagement with the desired fixation location, depending on the position along the longitudinal axis of anchor 500. Anchor 500 may further include additional protrusions 510 (e.g., splines, I-beams, barbs, knurls, threads, ribs, ridges, tines, teeth, wedges, fins, grooves, and any combination thereof) that may partially cover the outer surface of anchor body 502. For example, additional protrusions 510 may be utilized to produce a larger cross-section, as discussed above. Protrusions 508, 510 may extend outwardly from the outer surface of anchor body 502 in a direction non-parallel to the longitudinal axis of anchor body 502.

Anchor 500 may further include groove 512, which is defined by side walls 514, 516. Anchor body 502 may further define at least a portion of the depth of groove 512. Groove 512 may be positioned in close proximity to distal end 506. At least a portion of side walls 514, 516 and/or anchor body 502 may be radiused. Groove 512 may be dimensioned to accept at least one graft (e.g., four strand graft). Particularly, the graft (not shown) may be placed in close proximity to groove 512 while anchor 500 is inserted into the desired fixation location (e.g., through impaction). Anchor 500 may be inserted into the desired fixation location through only impaction. Protrusions 508, 510 at least partially secure anchor 500 to the desired fixation location and groove 512 may provide a resting location for the graft (not shown).

Although the present disclosure has been described with reference to exemplary implementations, the present disclosure is not limited by or to such exemplary implementations. Rather, various modifications, refinements and/or alternative implementations may be adopted without departing from the spirit or scope of the present disclosure.

1. A biodegradable anchor, comprising:
an anchor body having a longitudinal axis, a proximal end, a distal end, and an outer surface; and
a plurality of splines associated with the outer surface of the anchor body and extending in a direction non-parallel to the longitudinal axis of the anchor body,
wherein each of the plurality of splines defines a spline depth which is a distance between the outer surface of the anchor body and an outer surface of the spline; and
wherein the anchor is configured and adapted to be at least partially impacted into a desired fixation location.

2. (canceled)

3. The biodegradable anchor of claim 1, wherein at least one of the plurality of splines includes at least one protrusion extends from the at least one spline.

4. The biodegradable anchor of claim 1, wherein each of the plurality of splines twists from about the proximal end to about the distal end of the anchor body.

5. The biodegradable anchor of claim 1, wherein the plurality of splines comprises five helical splines associated with the outer surface of the anchor body.

6. The biodegradable anchor of claim 1, wherein the plurality of splines comprises six helical splines associated with the outer surface of the anchor body.

7. The biodegradable anchor of claim 1, wherein each of the plurality of splines has a twist angle of about 180 degrees to about 360 degrees.

8. The biodegradable anchor of claim 1, further comprising a mounting element relative to the proximal end of the anchor body, wherein the mounting element has a cross-section that is dimensioned larger than a cross-section of the anchor.

9. The biodegradable anchor of claim 1, further comprising a distal tip relative to the distal end, wherein the distal tip is selected from the group consisting of a conical tip, a cylindrical tip, a spherical tip, a loop tip, and any combination thereof.

10. The biodegradable anchor of claim 1, wherein the anchor is at least in part formed from a citrate-based (co)polyester.

11. The biodegradable anchor of claim 10, wherein the citrate-based (co)polyester is the polycondensation product of citric acid and/or citrate with a least one C₄ to C₁₂ alkane diol.

12. The biodegradable anchor of claim 11, wherein the citrate-based (co)polyester is poly(1,8-octanediol citrate).

13. The biodegradable anchor of claim 1, wherein the anchor is at least in part formed from a composite comprising a citrate-based polymer and a bioceramic.

14. The biodegradable anchor of claim 13, wherein the bioceramic is selected from the group consisting of hydroxyapatite and beta-tricalcium phosphate.

15. The biodegradable anchor of claim 1, further comprising a cannulation hole extending from the proximal end to about the distal end, wherein the cannulation hole is substantially parallel to the longitudinal axis.

16. The biodegradable anchor of claim 1, further comprising at least one cross hole extending from the outer surface of the anchor body in a direction non-parallel to the longitudinal axis.

17. The biodegradable anchor of claim 1, wherein at least one of the distal end, anchor body, and proximal end are configured and dimensioned to receive at least one graft.

18. The biodegradable anchor of claim 1, wherein the distal tip on the distal end further comprises a groove at least partially non-parallel to the longitudinal axis of the anchor body, wherein the groove is configured and dimensioned to receive at least one graft.

19. The biodegradable anchor of claim 1, wherein in at least two positions along the longitudinal axis of the anchor, the combined cross-section of the anchor body and the at least one protrusion is dissimilar.

20. A biodegradable anchor, comprising:
an anchor body having a longitudinal axis, a proximal end, a distal end, and an outer surface; and
a plurality of splines associated with the outer surface of the anchor body and extending in a direction non-parallel to the longitudinal axis of the anchor body,
wherein each of the plurality of splines defines a spline depth which is a distance between the outer surface of the anchor body and an outer surface of the spline;
wherein each of the plurality of spines twists from about the proximal end to about the distal end of the anchor body at a twist angle of about 180 degrees to about 360 degrees, and
wherein the anchor is configured and adapted to be at least partially impacted into a desired fixation location.
